(19) United States
(12) Patent Application Publication
(10) Pub. No.: US 2023/0307146 A1
(43) Pub. Date: Sep. 28, 2023

(54) Software that connects all health facilities, professions and recipients.

(71) Applicant: Mohammed Salah Al-Nimr, Tanta (EG)

(72) Inventor: Mohammed Salah Al-Nimr, Tanta (EG)

(21) Appl. No.: 18/114,477

(22) Filed: Feb. 27, 2023

Related U.S. Application Data
(60) Provisional application No. 63/322,263, filed on Mar. 22, 2022.

(51) Int. Cl. G16H 80/00; G16H 40/67; G16H 10/60

(52) U.S. Cl. CPC G16H 80/00; G16H 40/67; G16H 10/60

(57) ABSTRACT

A system and method for connecting all health facilities is described by giving each patient, health profession and health facility at least one unique universal predefined permanent ID e.g., face ID, fingerprint, retina pattern . . . etc.. Patient medical history and data of patients, related health professions and related health facilities are taken and saved on a database, then all data related to that particular patient can be viewed, some health professions can modify that data. Moreover, there is a data synchronizer which helps in the connection between different health platforms users and helps in synchronizing data from other health-related platforms, also robotic surgeries can be performed remotely, by artificial intelligence, or by artificial intelligence assistance, and there are lots of other artificial intelligence applications.

### BACKGROUND OF THE INVENTION

### 1). Field of the Invention

This invention relates to a computer system and method for saving patient-related data, making remote surgeries, and connecting all health facilities, professions and recipients.

### 2). Discussion of Related Art

Some existing health-related systems allow users to take patient history. In these software the taken data aren't well-organized i.e. the professional must take the medical history for each new patient from scratch or have to contact previous professionals after reference.

Health-related systems information also are little in most cases and not organized to build any artificial intelligence module.

Health-related systems information also are little in most cases and not organized so the retrospective studies based on these information are of no scientific value.

Some existing patient-related systems allow users to give and write a feedback about the health professional. In these software the given data aren't accurate as the patients or owners' in the case of non-humans in many cases are biased by the professional personality, appearance and the facility decoration . . . etc. not based on a scientific basis.

Some existing health-related systems allow users to view patient history. In these software the patient must have an external tool e.g., medical card, identity number or smart watch . . . etc. So it isn't of a value in most cases e.g., in death or in memory loss cases especially if the patient has nothing with him, also no universal communication can be made as the identity numbers aren't universal permanent IDs.

Some existing health-related systems allow users to make diagnoses based on artificial intelligence modules. In these software there is no thing to do in case of emergency especially with non-professionals. Moreover, these modules are based on very little data, so they aren't accurate.

Some existing health-related systems allow users to make video calls and conferences. In these software, there is no unique universal predefined permanent ID to guarantee that the professional is really licensed. Also, any video conference software can make these calls.

Some existing robotic surgical systems allow users to perform surgeries but on patient within the same place.

Health-related systems can't help patients to order their drugs from other countries based on their status.

### SUMMARY OF THE INVENTION

The invention provides a computer system related to health professions, facilities and patients or owners' in the case of non-humans that gives one or more unique universal predefined permanent ID or IDs for each patient e.g., face ID when taking patient medical information so by which patient's information can be accessed or modified by allowed professionals without the need to contact other health professionals who took or modified the patient history previously.

The invention also provides a method of modifying the identity numbers by adding a static code to each country's identity numbers in order to make them universal, the static code is unique for each place.

The invention also provides a method of connecting health facilities by giving one or more unique universal predefined permanent ID or IDs for each facility.

The invention also provides a method of connecting health professionals by giving one or more unique universal predefined permanent ID or IDs for each professional e.g., face ID, fingerprint, retina pattern . . . etc.

The invention also provides a method of connecting health facilities with health professionals by giving one or more unique universal predefined permanent ID or IDs for each professional e.g., face ID, fingerprint, retina pattern . . . etc. and giving one or more unique universal predefined permanent ID or IDs for each facility.

The invention also provides a method of connecting health professionals with health facilities by giving one or more unique universal predefined permanent ID or IDs for each professional e.g., face ID, fingerprint, retina pattern . . . etc. and giving one or more unique universal predefined permanent ID or IDs for each facility.

The invention also provides a method of connecting health professionals with health facilities and patients or owners' in the case of non-humans by giving one or more unique universal predefined permanent ID or IDs for each professional e.g., face ID, fingerprint, retina pattern . . . etc., giving one or more unique universal predefined permanent ID or IDs for each facility and giving one or more unique universal predefined permanent ID or IDs for each patient e.g., face ID, fingerprint, retina pattern . . . etc.

The invention also provides a method of connecting health facilities with health professionals and patients or owners' in the case of non-humans by giving one or more unique universal predefined permanent ID or IDs for each professional e.g., face ID, fingerprint, retina pattern . . . etc., giving one or more unique universal predefined permanent ID or IDs for each facility and giving one or more unique universal predefined permanent ID or IDs for each patient e.g., face ID, fingerprint, retina pattern . . . etc.

The invention also provides a method of connecting patients or owners' in the case of non-humans with health professionals and health facilities by giving one or more unique universal predefined permanent ID or IDs for each professional e.g., face ID, fingerprint, retina pattern . . . etc., giving one or more unique universal predefined permanent ID or IDs for each facility and giving one or more unique universal predefined permanent ID or IDs for each patient e.g., face ID, fingerprint, retina pattern . . . etc.

The invention also provides a method of connecting patients or owners' in the case of non-humans with health professionals by giving one or more unique universal predefined permanent ID or IDs for each professional e.g., face ID, fingerprint, retina pattern . . . etc., and giving one or more unique universal predefined permanent ID or IDs for each patient e.g., face ID, fingerprint, retina pattern . . . etc.

The invention also provides a method of connecting patients or owners' in the case of non-humans with health facilities by giving one or more unique universal predefined permanent ID or IDs for each facility and giving one or more unique universal predefined permanent ID or IDs for each patient e.g., face ID, fingerprint, retina pattern . . . etc.

The invention also provides a method of rating health professionals in an accurate manner i.e. by rating the number of patients for each health professional and their progress rate.

The invention also provides a method of confirming health professionals in an accurate manner i.e. by asking for details about their license from their authorizer.

The invention also provides a method of recommending a health professional for patients in an accurate manner i.e. by rating the number of patients for each health professional and their progress rate and recommendation based on rating, location, . . . etc.

The invention also provides a method of rating health facilities in an accurate manner i.e. by rating the number of patients for each health facility and their progress rate.

The invention also provides a method of recommending health facilities for patients or owners' in the case of non-humans in an accurate manner i.e. by rating the number of patients for each health facility, their progress rate then the recommendation is based on rating, location . . . etc.

The invention also provides a method of accurate retrospective studies performance by collecting a variety of worldwide data and using an artificial intelligence module to perform studies.

The invention also provides a method of artificial intelligence to build a module that can publish a variety of studies about almost everything related to health studies based on given data.

The invention also provides a method of getting important information about cases in case of emergency, even without the need for any external identity.

The invention also provides a method of viewing basic information of cases in case of emergency especially for memory loss cases with no identity or death with no identity cases.

The invention also provides a method of dealing with cases in case of emergency, even for non-specialized.

The invention also provides a method of epidemic and/or pandemic data collection that help in quick reactions.

The invention also provides a method of performing a remote robot surgery by professionals.

The invention also provides a method of performing a remote robot surgery by artificial intelligence.

The invention also provides a method of performing a remote robot surgery by a combination of artificial intelligence and professionals.

The invention also provides a method of data synchronization between different health platforms.

The invention also provides a method of connection between users using different health platforms.

The features and advantages described in this summary and the following detailed description are not all-inclusive. Many additional features and advantages will be apparent to one of ordinary skill in the art in view of the drawings, specifications, and claims.

### BRIEF DESCRIPTION OF THE DRAWINGS

Exemplary embodiments of the present disclosure will become more fully apparent from the following description and appended claims, taken in conjunction with the accompanying drawings. It should be understood that the drawings illustrate only exemplary embodiments, and therefore, do not limit the scope of the disclosure. The exemplary embodiments of the invention will be described with additional specificity and detail through use of the accompanying drawings in which:

FIG. 1 is a block diagram illustrating the different modules inside the medical communication system, configured according to one embodiment of the present disclosure.

FIG. 2 is a signaling diagram illustrating a method for communications between health facilities, health professionals, and/or patients or owners (in case of non-humans), according to one embodiment of the present disclosure.

FIG. 3 illustrates an exemplary patient profile page as accessed by the patient himself or by the owner (in case of non-humans), according to one embodiment of the present disclosure.

FIG. 4 illustrates an exemplary patient health-related professional profile page as viewed by the patient himself or by the owner (in case of non-humans), according to one embodiment of the present disclosure.

FIG. 5 illustrates an exemplary artificial intelligence module-based page that diagnoses cases, recommend professionals or help in the treatment plan, and exemplary output of the professional recommendation, according to one embodiment of the present disclosure.

FIG. 6 illustrates the output of the exemplary artificial intelligence module-based page that diagnoses cases or helps in differential diagnosis, according to one embodiment of the present disclosure.

FIG. 7 illustrates an exemplary health professional profile page as accessed by the health professional himself, according to one embodiment of the present disclosure.

FIG. 8 illustrates an exemplary patient creation page as accessed by the health professional, according to one embodiment of the present disclosure.

FIG. 9 illustrates an exemplary patient information viewing, modifying and record adding page as accessed by the permitted health professional, according to one embodiment of the present disclosure.

FIG. 10 illustrates an exemplary retrospective study performing page as accessed by a health professional, according to one embodiment of the present disclosure.

FIG. 11 illustrates an exemplary published retrospective study page as published by an artificial intelligence module, according to one embodiment of the present disclosure.

FIG. 12 illustrates an exemplary emergency login page as accessed by a health professional, according to one embodiment of the present disclosure.

FIG. 13 illustrates an exemplary emergency patient profile page as accessed by a health professional, according to one embodiment of the present disclosure.

FIG. 14 is a functional diagram illustrating respective methods for synchronizing data between an exemplary health-related software other than ours and the one in our invention, according to one embodiment of the present disclosure.

FIG. 15 is a functional diagram illustrating respective methods for routing data between users of different exemplary health-related software other than ours via our invention, according to one embodiment of the present disclosure.

FIG. 16 illustrates an exemplary published retrospective study page as published by an artificial intelligence module and a way to order the active ingredient, according to one embodiment of the present disclosure.

FIG. 17 illustrates an exemplary emergency login page as accessed by a non-health professional, according to one embodiment of the present disclosure.

FIG. 18 is a functional diagram illustrating respective methods for connection between exemplary robotic surgical systems users through the software in our invention, according to one embodiment of the present disclosure.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

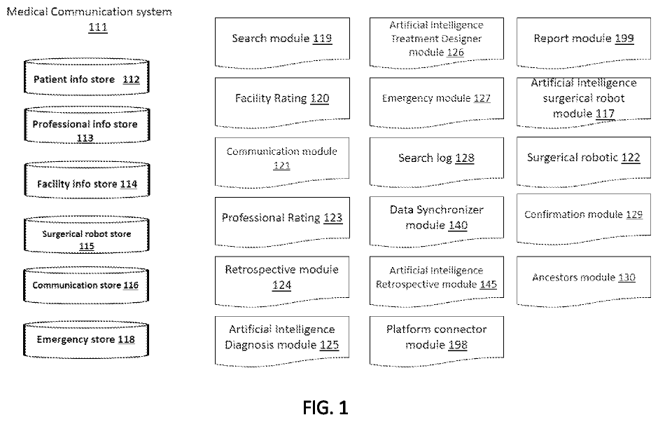

Those of skill in the art will appreciate that the medical communication system 111 may contain other modules that are not described herein and that the particular components seen in FIG. 1 are for illustrative purposes only, also that medical communication system 111 may comprise more, fewer, or different components than those seen in FIG. 1 as needed or desired. In addition, conventional elements, such as firewalls, authentication systems, payment processing systems, network management tools, load balancers, booking system, chatting system, request system, facility managing system and so forth are not shown as they are not material to the invention.

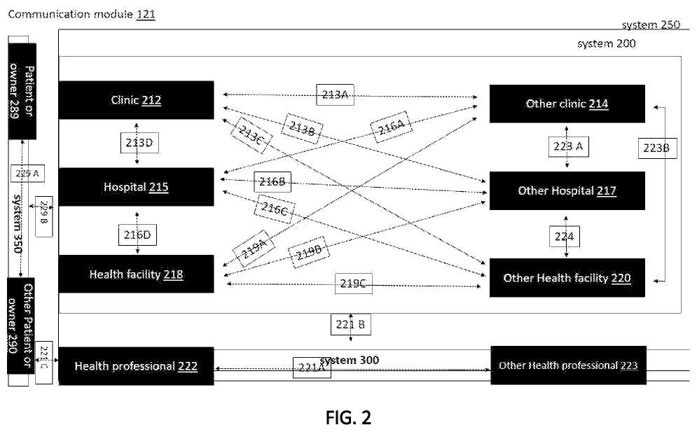

Referring now to FIG. 1 and FIG. 2, in one embodiment of the present invention. FIG. 1 and the other figures use like reference numerals to identify like elements. A letter after a reference numeral, such as “213A,” indicates that the text refers specifically to the element having that particular reference numeral. A reference numeral in the text without a following letter, such as “213,” refers to any or all of the elements in the figures bearing that reference numeral (e.g., “213” in the text refers to reference numerals “213A”, “213B”, “213C” and/or “213D” in the figures). Moreover, the word professional refers to human professional, robot and/or the combination. Also the word patient refers to human patient, non-human patient, the owner of the non-human patient, and/or any combination of them.

In one embodiment the medical communication system 111 comprises a patient info store 112, a professional info store 113, a facility info store 114, a surgical robot store 115, a communication store 116, an artificial intelligence surgical robot module 117, an emergency store 118, a search module 119, a facility rating 120, a communication module 121, a robotic surgical module 122, a professional rating 123, a retrospective module 124, an artificial intelligence diagnosis module 125, an artificial intelligence treatment designer module 126, an emergency module 127, a search log 128, a confirmation module 129, an ancestors module 130, a data synchronizer module 140, an artificial intelligence retrospective module 145, a platform connector module 198 and a report module 199. Those of skill in the art will appreciate that the system 111 may be implemented using a single computer, or a network of computers, including cloud-based computer implementations. The computers are preferably server class computers including one or more high-performance CPUs and 16 or more of main memory, and running an operating system such as Linux or variants thereof. The operations of the system 111 as described herein can be controlled through either hardware or through computer programs installed in non-transitory computer storage and executed by the processors to perform the functions described herein. The various stores (e.g., patient info store 112, professional info store 113, etc.) are implemented using non-transitory computer readable storage devices, and suitable database management systems for data access and retrieval. The system 100 includes other hardware elements necessary for the operations described here, including network interfaces and protocols, input devices for data entry, and output devices for display, printing, or other presentations of data.

The patient info store 112 persistently stores data related to patients that were added by professionals in the medical communication system 111, and is one means for performing this function. Information about patients includes patient personal information such as name, email address, location, phone number, emergency phone, emergency mail, gender, date of birth, personal description, education, work, pictures, and the like. Moreover, patient info store 112 stores additional information such as medical history. Each patient is assigned one or more unique universal predefined ID. The unique universal predefined permanent ID may be numerical, alphabetic, combination or any other type of data such as fingerprint, retina pattern, face print, DNA and the like.

The professional info store 113 persistently stores data related to users who are in a health profession, and is one means for performing this function. Information about professionals includes professional personal information such as name, email address, location, phone number, gender, date of birth, personal description, education, degree, pictures, and the like. Furthermore, the professional info store 113 may store additional information such as professional rating 123, and facility info store 114. Each professional is assigned one or more unique universal predefined permanent ID. The unique universal predefined permanent ID may be numerical, alphabetic, combination or any other type of data such as fingerprint, retina pattern, face print, DNA and the like.

The facility info store 114 persistently stores data related to health facilities that were added in the medical communication system 111, and is one means for performing this function. Information about facilities includes facility information such as name, email address, location, phone number, hotline, departments, specialties, and the like. Moreover, facility info store 114 stores additional information such as facility rating 120. Each facility is assigned one or more unique universal predefined permanent ID e.g., employer identification number and their data is confirmed through confirmation module 129. The facility info store 114 also generates the unique universal predefined permanent ID by modifying the employer identification number e.g., by adding the country phone code to the employer identification number which will make it a unique universal predefined permanent ID.

The surgical robot store 115 persistently stores data from operations performed through the medical communication system 111 by professionals, and is one means for performing this function. Information about operations includes patient's medical history such as sex, age diseases, x-rays, laboratory tests, and the like. Moreover, surgical robot store 115 stores additional information such as action taken during the operation, success rates of the operation, and the like. Each robot, professional, and/or facility is assigned one or more unique universal predefined permanent ID.

The communication store 116 persistently stores data related to the communication module 121 in the medical communication system 111, and is one means for performing this function. Information about communication includes facility information such as name, email address, location, phone number, hotline, departments, specialties, and the like. Also the information includes patient information such as name, email address, phone number, and the like. Moreover, communication store 116 stores additional information such as messages, comments from facilities, patients or both.

The artificial intelligence surgical robot module 117 allows patients to have operations performed by artificial intelligence based on data from the surgical robot store 115 and data from the patient info store 112, and is one means for performing this function.

The emergency store 118 persistently stores data related to emergency module 127 in the medical communication system 111, and is one means for performing this function. Information about emergency store 118 includes patient information such as name, email address, phone number, emergency phone, emergency mail, relatives, medical information that is important in emergency cases such as allergies, previous operations, chronic diseases . . . etc., and the like, also emergency store 118 include information about dealing with the case even for non-professionals. Moreover, emergency store 118 stores additional information such as professional info store 113, facility info store 114, and patient info store 112.

The search module 119 receives an input query from a patient and returns a list of facility and professional listing that best match the input query, and is one means for performing this function. The search query includes search parameters regarding the patient's symptoms, for example, they urinate a lot, often at night, are very thirsty, lose weight without trying, are very hungry, have blurry vision, have numb or tingling hands or feet, feel very tired, have very dry skin, and the like; also the patient's preferences, such as facility type, price range, and the like, and patient location . . . etc. The search module 119 then retrieves all the listing that match the search query. In one embodiment, Boolean matching is used for parameters such as location and symptoms, and price range, with additional parameters used to further filter the results.

In some embodiments, the search module 119 ranks the returned search results based on a ranking score. The ranking score is a function of a number of factors, such as price, professional rating 123, facility rating 120, location, listing, or a combination thereof. The ranking function be implemented as a linear combination of the individual factors, where each factor is represented as a scaled variable indicating a degree of match (e.g., 1 of an exact match of the underlying search parameter, 0.5 for a partial or near match), and weighted with a weight to reflect the importance of the factor. Typically, the professional rating 123, the facility rating 120 and the location are highly weighted, and prices are lesser weighted, but the particular weights are a design decision for the system administrator.

The facility rating 120 provides a numerical representation of the success rate of the previous treatment in a health facility. The facility rating 120 can be based on the previous treatment upcoming of the professionals of the facility. For example, each time a professional in the facility performs an operation, describes a treatment plan . . . etc. Its success rate is based on the upcoming of the patient so the complete recovery is considered a full rate while the persistence of the problem is assigned as a failure then the average of rate of all professionals while working in the facility is considered the facility rating i.e. If the full rate is 5 and failure is zero and the facility has three professionals the first professional had the full rate ten times and had failed two times while working in the facility then their rate would be (10×5+2×0)/12 which is 4.16/5 which is the sum of the numbers divided by how many numbers are in the list and the rating is from the full rate number, the second professional had the full rate two times and had failed one time while working in the facility then their rate would be (2×5+1×0)/3 which is 3.3/5, and the third professional had the full rate six times with no fails while working in the facility then their rate would be (6×5)/6 which is 5/5, so the facility rating 120 would be (4.16+3.3+5)/3=4.15 from 5 which is the average of the facility professionals while working in the facility i.e. The sum of the facility professional rating divided by how many facility professional rating are in the list.

The communication module 121 allows all types of communication between all health facilities such as a clinic 212 with another clinic 214 through 213A connection pathway, a clinic 212 with a hospital 217 through 213B connection pathway, a clinic 212 with any other health facility 220 through 213C connection pathway, a hospital 215 with a clinic 214 through 216A connection pathway, a hospital 215 with another hospital 217 through 216B connection pathway, a hospital 215 with any other health facility 220 through 216C connection pathway, a health facility 218 with a clinic 214 through 219A connection pathway, a health facility 218 with a hospital 217 through 219B connection pathway, a health facility 218 with any other health facility 220 through 219C connection pathway, or any combination of them through system 200, the communication module 121 also allows communication between any health professional 222 and any other health professional 223 through 221A connection pathway through system 300, also the communication module 121 allows all types of communication between system 200 or any part of the system 200 with system 300 or any part of system 300 through 221B connection pathway, the communication module 121 allows also all types of communication between patient or owner 289 with any other patient or owner 290 through 229A connection pathway through system 350, the communication module 121 allows also all types of communication between system 350 or any part of the system 350 with system 300 or any part of system 300 through 221C connection pathway, and the communication module 121 allows also all types of communication between system 350 or any part of the system 350 with system 200 or any part of system 200 through 229B connection pathway. Each professional in the communication module 121 is assigned to their unique universal predefined permanent ID, also each patient is assigned to their unique universal predefined permanent ID. The unique universal predefined permanent ID of the patient and of the professional is based on the one in the medical communication system 111 data. While not specifically shown in the figures, the connection pathways aren't limited to that mentioned herein i.e. they may become between more or fewer users or may use systems in apart e.g., the system may function only by connecting patients with each other with no professional or facility, professionals with each other, professionals with patients with no facility . . . etc, also the system may contain other users' types. The connection pathways are connecting via one or more public and/or private communications networks, as is known in the art. Such networks may comprise, for example, the internet or other such IP network capable of communicating information in data packets, any of a variety of wireless communications networks, or any combination thereof.

The robotic surgical module 122 allows the professionals to perform remote surgeries by the robots in the operating theater, and is one means for performing this function. Each professional, robot, patient and facility is given a unique universal predefined permanent ID and all actions taken in the operation are stored within the surgical robot store 115.

In some embodiments the robotic surgical module allows performing the procedure completely by artificial intelligence or by a combination of both professionals and artificial intelligence.

The professional rating 123 provides a numerical representation of the success rate based on previous treatment of a professional, a robot, and/or a combination. The professional rating 123 can be based on the the previous treatment upcoming of the professional. For example, each time a professional performs an operation, describes a treatment plan . . . etc., its success rate is based on the upcoming of the patient, so the complete recovery is considered a full rate while the persistence of the problem is assigned as a failure then the average of rate is considered the professional rating i.e. If the full rate is 5 and failure is zero, and the professional had the full rate four times and had failed two times then their rate would be (4×5+2×0)/6 which is 3.33/5 which is the sum of the numbers divided by how many numbers are in the list and the rating is from the full rate number.

The retrospective module 124 allows health professionals to perform studies on relations between different factors that lead to valued information about these factors, and is one means for performing this function. The retrospective module query includes search parameters regarding the geographic location, the particular time, the particular sex, the particular ages, and the like, any combination of them, or with none of them without encroaching on patients' privacy. By way of example only, if a professional wants to conduct a study on the relationship between aspartame and colorectal cancer in USA female white teenagers in 2020, then the health professional is asked to enter the data related to the study, which will be aspartame and colorectal cancer in the relation box, the USA will be selected from the map, the sex checkbox will be female, the race will be white, the age range will be 13-19, and the time will be 2020, then after confirmation the system will fetch all related data and perform a study with all statistics based on all patients' data without encroaching on patients' privacy.

The artificial intelligence diagnosis module 125 receives an input query from a patient or a health professional and returns a list of the differential diagnosis listing that best matches the input query, and is one means for performing this function. The diagnosis query includes diagnosis parameters regarding the patient's symptoms, such as they urinate a lot, often at night, are very thirsty, lose weight without trying, are very hungry, have blurry vision, have numb or tingling hands or feet, feel very tired, have very dry skin, and the like; also the patient's related reports, such as laboratory tests, x-rays, and the like. The artificial intelligence diagnosis module 125 then retrieves all the listing that match the diagnosis query. In one embodiment, additional parameters are required to be used to further filter the results in order to reach the definitive diagnosis. For example, with symptoms as they urinate a lot, often at night, are very thirsty, lose weight without trying, are very hungry, have blurry vision, have numb or tingling hands or feet, feel very tired, and have very dry skin the differential diagnoses will have hypercalcemia, diabetes and b vitamins deficiency. Additional parameters will be required, which will be HBA1C, CBC, free and ionized calcium tests in this case; then the definitive diagnosis will be reached based on the available information.

The artificial intelligence treatment designer module 126 receives the definitive diagnosis from the artificial intelligence diagnosis module 125 or from the health professional and returns a list of the treatments listing that best match the input query, and is one means for performing this function. The treatment query includes treatment parameters regarding the patient's definitive diagnosis, the patients' information and the patient history. The artificial intelligence treatment designer module 126 then retrieves all the listing that match the treatment query. The artificial intelligence treatment designer module 126 also recommends a treatment plan for every case based on their medical history such as allergies, diet, age . . . etc. And based on the personal information like their financial status and their occupation.

The emergency module 127 allows professionals to view urgent emergency information about any patient by entering the patient's unique universal predefined permanent ID without any authorizations, and is one means for performing this function. The emergency information includes some of the medical history information such as allergies, previous operations, and chronic diseases. The emergency information also includes some of the personal information like name, age, emergency phone, and relatives. The emergency module 127 also allows professionals to add data to patients' emergency store 188. The emergency module 127 also informs the patient that his information was viewed by a health professional in the emergency mode, and the information about the health professional who viewed their emergency information is sent to the patient which may include name, facility information, location, and the like to help in making patient privacy safe. Moreover, the emergency module 127 allows non-professionals to view urgent emergency information about patients by entering the patient's unique universal predefined permanent ID without any authorizations, and is one means for performing this function. The emergency information includes some of the medical history such as allergies, previous operations, and chronic diseases. The emergency information also includes some of the personal information like name, age, emergency phone, and relatives. The emergency information also includes information and instructions to deal with the case, even for non-professionals 1711.

The search log 128 keeps a record of all search queries performed in the medical communication system 111, and is one means for performing this function. Embodiments maintain the information in a database or other type of data repository. Every search query is associated with a patient and includes information about the search parameters and the set of listing obtained by the search module that match the query. Some embodiments of the search log 219 also store information regarding the actions taken by the patient after receiving the list of possible facilities and professionals. For example, the search log may maintain information about which listings the user clicked or viewed, and which listing the patient requested booking.

The confirmation module 129 sends license data to the authorizer facility to make sure the license is valid and no scam, it also makes sure that the authorizer facility is really found and legally licensed by validating data related to them e.g., the data taken from patients are validated through checking that patients had visits with different professionals and facilities which should differ from patient to the other and share the facility under check. Also the address can be confired by asking random professionals and/or patients to check in there. Moreover, the facility information can be modified after some of their professionals submit an order e.g., 30% of professional ask to change the identifier of the facility as it is changed and their admins no longer have access. It is to know the percentage of professionals who have to submit order varies, and the system administrators may decide different ways to allow changing the identifier e.g., authorizer request.

The data synchronizer 140 allows medical communication system 111 to synchronize data from other platforms that are in relation to the health field, and is one means for performing this function. The data synchronizer 140 also modify that data so that every professional will have one or more unique universal predefined permanent ID and every patient will have one or more unique universal predefined ID, in order to organize the data from the different platforms and in order to organize the data from the different professionals for the same patients. The medical communication system 111 decide that the data on different platforms is for the same patient either by a request or by common active mail, active phone number and the like. Also the medical communication system 111 decides that the data on different platforms is for the same professional either by a request or by a common active mail, active phone number and the like. The unique universal predefined permanent ID of the patient and of the professional is based on the one in the medical communication system 111 data.

The ancestors module 130 allows medical communication system 111 to understand familial relations between users, especially the health care recipients based on data obtained from the patient info store 112 and from the professional info store 113 such as DNA, and is one means for performing this function. The ancestors module 130 also allows users to find their relatives and/or ancestors under predefined standards in order to save privacy.

The artificial intelligence retrospective module 145 performs retrospective studies on its own, and is one means for performing this function. Embodiments of the artificial intelligence retrospective module 145 use artificial intelligence, statistical models and predictive models. In other embodiments, other artificial intelligence algorithms, such as neural network, random forest, logistic regression, time-series, clustering, decision trees, any other supervised learning algorithms, or any other unsupervised learning algorithms may be used to build the statistical or the predictive model. On a relation base the artificial intelligence retrospective module 145 calculates the relation between any two or more factors within the data, when the relationship is of a value e.g., greater than 70% the model starts to publish the study, and predicts possible new ways in preventive medicine or new treatments. By way of example only, if the artificial intelligence retrospective module 145 found that there is a relation between aspartame and colorectal cancer treatment as in 80% of cases who used aspartame and were diagnosed with colorectal cancer previously were cured, then the artificial intelligence retrospective module 145 will publish a study with these results, and the predictive model will predict that aspartame is a good treatment for colorectal cancer if combined with aloe vera as the curing rate was up to 95% in people who used the combination of aspartame and aloe vera twice a day for six months.

The platform connector 198 allows users' data to be merged even they were using different platforms that is in a relation to the health field, and is one means for performing this function. The platform connector 198 gives every user their unique universal predefined permanent ID even if they are using other platforms at the same time, their data on the other platforms are synchronized via the data synchronizer 140 immediately, and they are allowed to communicate with each other through the platform connector 198 without the need to be signed into the same platform. The unique universal predefined permanent ID of the patient and of the professional is based on the one in the medical communication system 111 data.

The report module 199 allows patient users to report any uncomfortable diagnosis or information to be known by all related health professionals, and is one means for performing this function. The report module 199 uses algorithms to reflect the medical importance of this information by weighting them and deleting the lightly weighted information from the users' interface. Typically, emergency medical information, and chronic diseases are highly weighted, and aesthetics are lesser weighted, but the particular weights are a design decision for the system administrator. Moreover, the report module 199 reports the emergency department of one or more of the nearest health facilities in emergency cases, also report module 199 informs the detail about the case to its emergency contacting individuals.

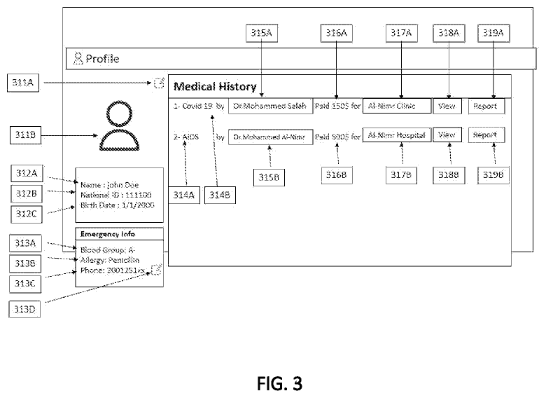

FIG. 3 shows an exemplary embodiment of the user interface for a patient to view their information, or modify modifiable information related to patient info store 112. The exemplary user interface includes means for the patients to view their profile image 311, personal information 312, emergency information 313, medical records 314, related health professionals 315, costs 316, and related health facilities 317. The exemplary user interface also includes means for the patient to modify their personal information that can be changed such as profile image 311A, and emergency phone 313D. The exemplary user interface also includes means for the patient to view full details of their health professional 315, health facility 317, and medical records 318. The exemplary user interface includes also a means for the patient to report their unpleasant medical records 319.

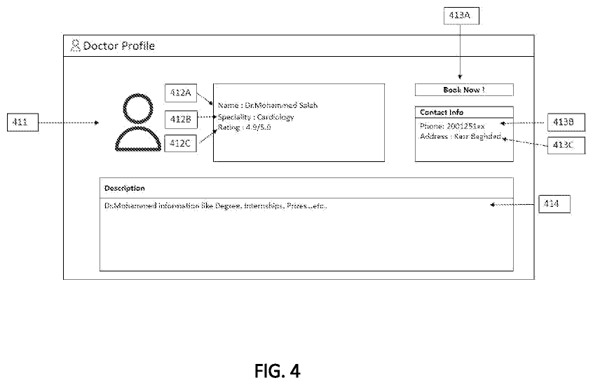

FIG. 4 shows an exemplary embodiment of the user interface for a patient to view health professional information related to professional info store 113. The exemplary user interface includes means for the patients to contact health professionals and/or view the health professional profile image 411, personal information such as name 412A, medical information such as specialty 412B, and rating 412C, contact information 413, other health professional's information 414. The health professional's rating in the user interface 412C includes rating information based on the professional rating 123.

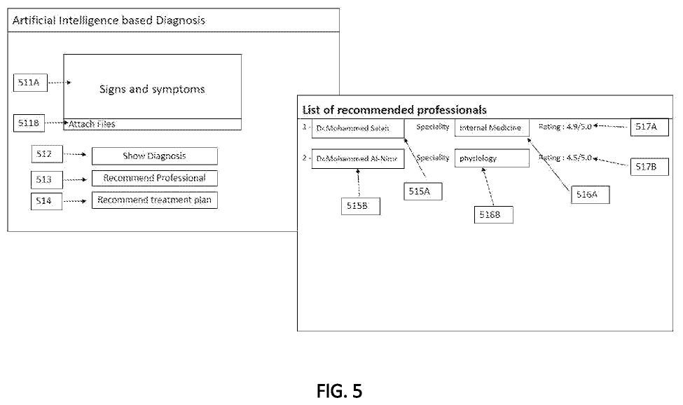

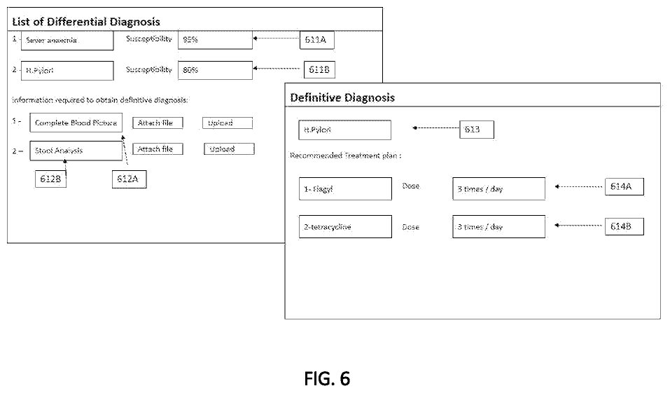

FIG. 5 and FIG. 6 show an exemplary embodiment of the user interface for users to use artificial intelligence to help in diagnosing the case based on information from the artificial intelligence diagnosis module 125. The exemplary user interface includes means for user to enter signs and symptoms 511A, and attach files such as laboratory examinations and x-rays 511B. As a result, the artificial intelligence diagnosis module 125 retrieves all the listings of differential diagnosis that match the diagnosis query with their susceptibility percentage 611. In one embodiment, additional parameters 612 are required to be used to further filter the results in order to reach the definitive diagnosis 613. The exemplary user interface includes means 513 that recommend professionals 515 according to their rating 517 mentioning their specialty 516, and also means 514 that recommend the treatment plans 614.

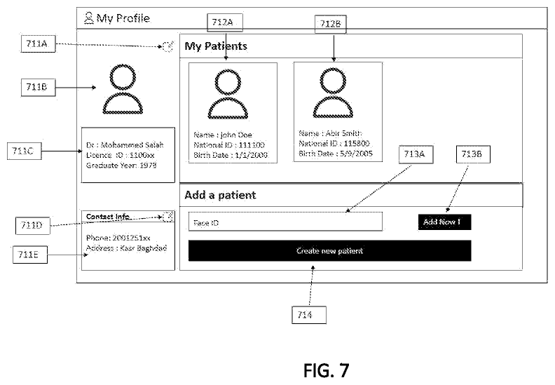

FIG. 7 shows an exemplary embodiment of the user interface for health professionals to view their information such as 711. The exemplary user interface includes means for the health professional to view their patients 712, means to add new patients either by one of their unique universal predefined permanent ID that relates to the medical communication system 111 if they exist on the patient info store 112 which is the face ID in the exemplary FIG. 713, or to create new patient 714 if not exist on the patient info store 112, and means to modify modifiable data such as profile image 711A, or contact information 711D.

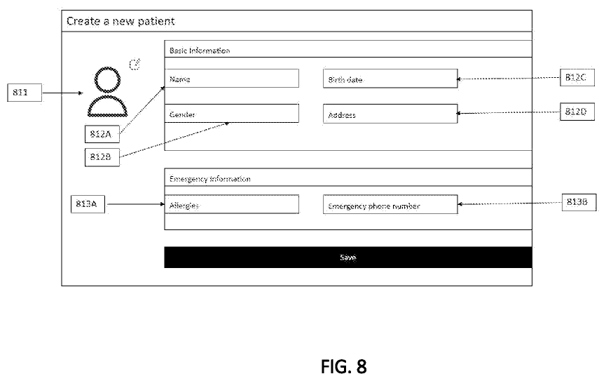

FIG. 8 shows an exemplary embodiment of the user interface for health professionals to create a new patient by adding basic information to the database such as basic information like their image 811, name 812A, gender 812B, birth date 812C, Address 812D . . . etc. Which will be related mainly to patient info store 112, and by adding emergency information which will be related to both patient info store 112, and emergency store 118. Some information from the patient info store 112 may be shared with emergency store 118.

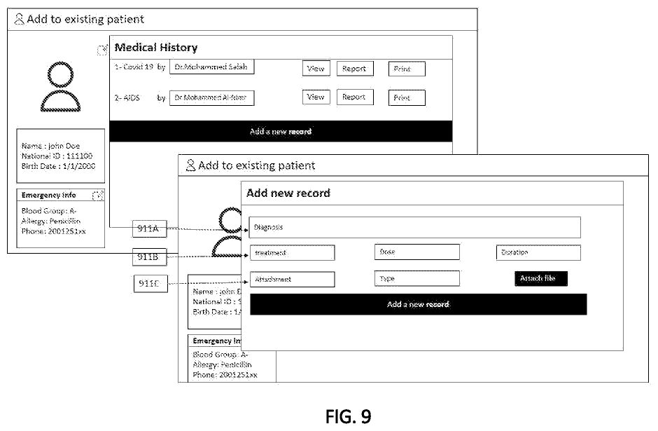

FIG. 9 shows an exemplary embodiment of the user interface for health professionals to add records to the existing patients by adding diagnosis 911A, treatment plan 911B, and/or attachments like laboratory tests and x-rays 911C.

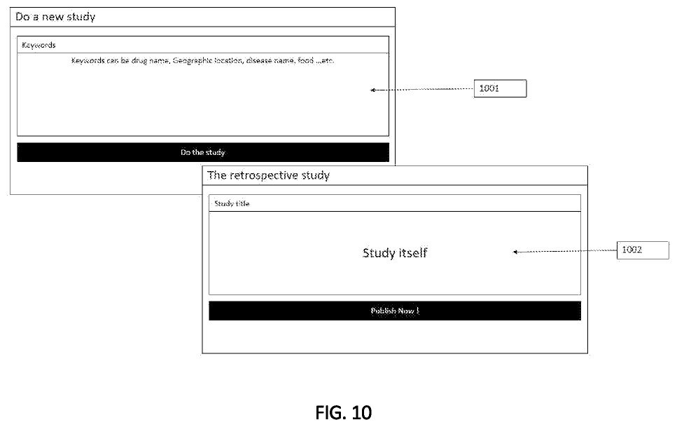

FIG. 10 shows an exemplary embodiment of the user interface for health professionals to enter keywords for a retrospective study 1001 and review the study results 1002 provided by the retrospective module 124.

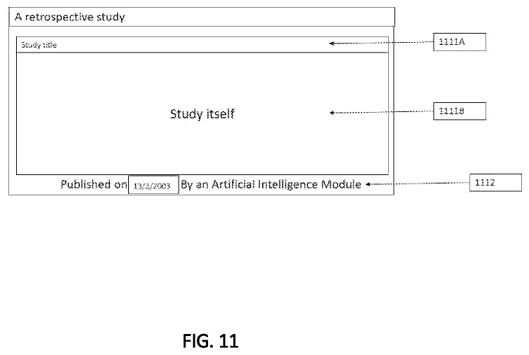

FIG. 11 shows an exemplary embodiment of a retrospective study 1111 provided by the artificial intelligence retrospective module 145. The study must have a title 1111A, a body 1111B, and a publication date and time 1112 at least.

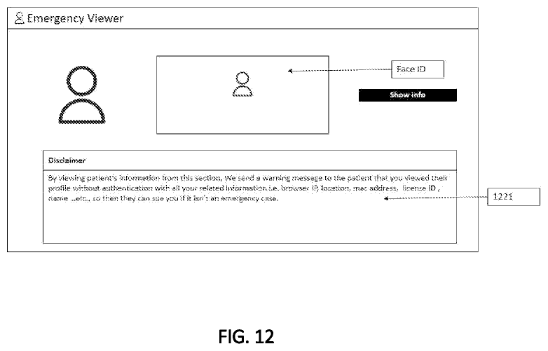

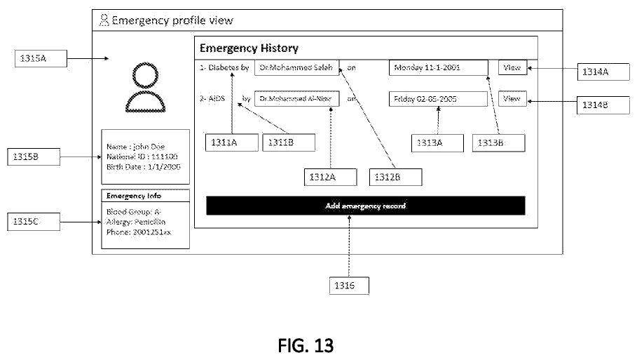

FIG. 12 and FIG. 13 show an exemplary embodiment of the user interface for health professionals to enter the unique universal predefined permanent ID for the patient in the drawing case it is the face ID and viewing their emergency information provided by the emergency module 127 such as some personal information 1315, emergency medical history 1311, emergency medical history related professionals 1312, emergency medical history date and time 1313. The exemplary user interface includes also a means to view the full detailed medical record 1314, and a means to add emergency records 1316.

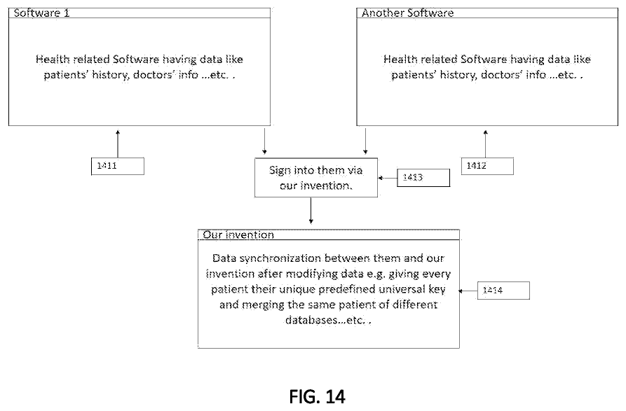

Referring now to FIG. 14, there is shown the synchronizing system architecture adapted to support one embodiment of the present invention. The medical communication system 111 represents communication between different health-related platforms 1413 such as the first platform 1411, and second platform 1412. Data Synchronizer module 140 then synchronizes data from both platforms 1411 and 1412, modify them by adding the unique universal predefined permanent ID to both health professional and patient, and rearranges them in order to have valued data about both health professionals and patients. The unique universal predefined permanent ID of the patient and of the professional is based on the one in the medical communication system 111 data.

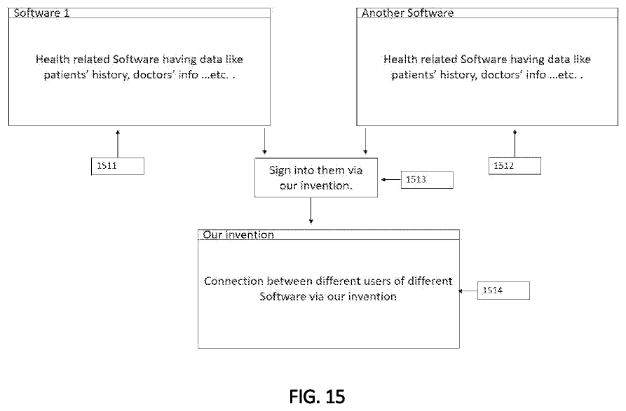

FIG. 15, there is shown the cross-platform communication system architecture adapted to support one embodiment of the present invention. The medical communication system 111 represents a cross-platform user communication 1513 between different health-related platforms such as first platform 1511, and second platform 1512. Platform connector module 198 then allows users on different platforms to communicate and share their data without needing to be on the same platform. For example if the first professional is using the first platform 1511 to record patients and the second professional is using the second platform 1512 to record patients, they can share their patient data with each other via the cross-platform user communication 1513 system.

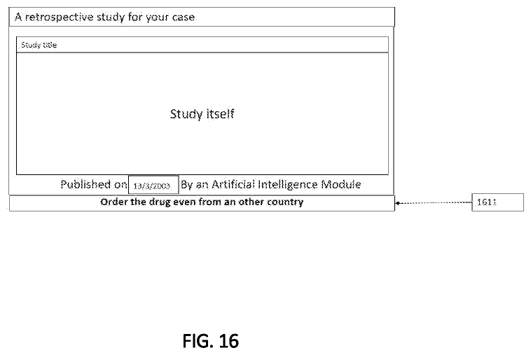

FIG. 16 shows an exemplary embodiment of a retrospective study provided by the artificial intelligence retrospective module 145. The artificial intelligence retrospective module 145 allows users to order the ingredient within the study even from other countries 1611.

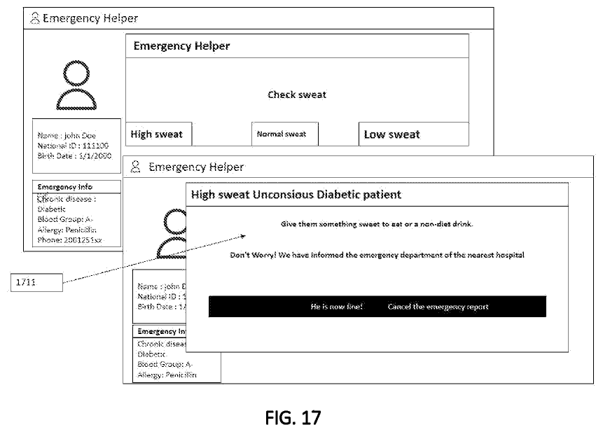

FIG. 17 show an exemplary embodiment of the user interface for non-health professionals to enter the unique universal predefined permanent ID for the patient in the drawing case it is the face ID and viewing their emergency information provided by the emergency module 127 such as some personal information 1315, emergency medical history 1311, emergency medical history related professionals 1312, emergency medical history date and time 1313. The exemplary user interface also includes means to deal with the case even for non-professionals 1711 provided by the emergency module 127 and reporting status to the emergency department of one or more of the nearest health facilities provided by the report module 199. Moreover, the report module 199 also informs the emergency contacting individuals.

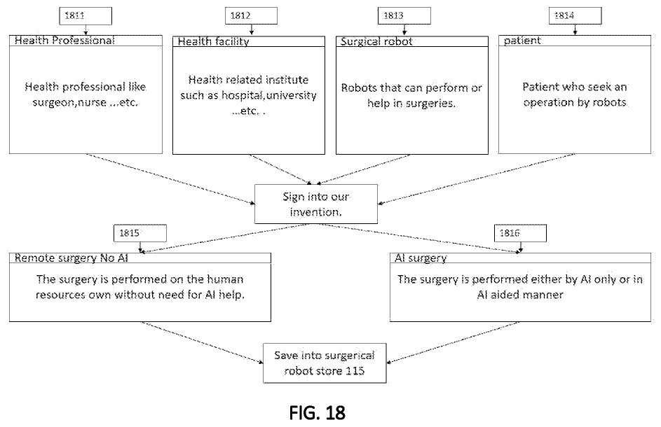

Referring now to FIG. 18, there is shown the remote robot surgery system architecture adapted to support one embodiment of the present invention. The medical communication system 111 represents communication between different health professionals 1811, different health facilities 1812, different surgical robots 1813, and patients 1814. Robotic surgical module 122 then synchronizes data from between different health professionals 1811, different health facilities 1812, different surgical robots 1813, and patients 1814, and allows connections between all of them in order to perform robotic surgeries by professionals 1815, artificial intelligence, or combination of them 1816, then all procedures are saved to the surgical robot store 115. The connection between different health professionals 1811, different health facilities 1812, different surgical robots 1813, and patients 1814 is mainly based on the unique universal predefined permanent ID of the patients, the professionals, the facilities, and the surgical robots reverted from the one in the medical communication system 111 data.

1. A computer implemented method, comprising: giving each health-related individual a unique universal predefined permanent ID.

2. The computer implemented method of claim 1, wherein giving each health-related facility a unique universal predefined permanent ID;
allowing the connection between any health-related individual user with any other;
allowing the connection any health-related individual user with any health facility;
allowing the connection between any health facility with any other health facility;
training a predictive computer model based on values of the health status of the patients, their personal information like gender, family history, geographic location, age . . . etc and their outcome to discover new drugs;
training a predictive computer model based on values of the health status of the patient, their personal information like gender, family history, geographic location, age . . . etc and their outcome to discover new uses of existing drugs;
training a predictive computer model based on values of the health status of the patients, their personal information like gender, family history, geographic location, age . . . etc, their personal information and their outcome to rate the existing drugs;
training a predictive computer model based on values of the health status of the patient and their outcome to rate the health professional;
training a predictive computer model based on values of the health status of the patients, their financial status, their geographic location and their outcome to recommend a suitable health professional for the patient;
training a predictive computer model based on values of the health status of the patient and their outcome to rate the health facilities;
training a predictive computer model based on values of the health status of the patients, their financial status, their geographic location and their outcome to recommend a suitable health facility for the patient;
training a predictive computer model based on values of the health status of the patient and their outcome to make artificial intelligence based diagnosis software;
training a predictive computer model based on values of the health status of the patients, their personal information like gender, family history, geographic location, age . . . etc, their personal information and their outcome to make retrospective studies;
training a predictive computer model based on values of the unique universal predefined permanent ID of the users e.g. the DNA, to understand familial relations between users.

3. The computer implemented method of claim 1, wherein the health facilities and professionals are rated based upon scientific basis.

4. The computer implemented method of claim 1, wherein some of the patients' data can be accessed without authorization but under law standards in case of emergency.

5. The computer implemented method of claim 1, wherein the Synchronizer module allows connection between users on different medical platforms.

6. The computer implemented method of claim 1, wherein the robotic surgical module allows performing remote robotic surgeries even from other countries.

7. The computer implemented method of claim 1, wherein training the artificial intelligence computer model based on values of robotic surgical surgeries to assist in and perform surgeries.

8. A system comprising: a non-transitory computer-readable medium with instructions encoded thereon; and one or more processors configured to, when executing the instructions, perform operations of:
giving each health-related individual a unique universal predefined permanent ID.

9. The system of claim 8, wherein giving each health-related facility a unique universal predefined permanent ID;
allowing the connection between any health-related individual user with any other;
allowing the connection any health-related individual user with any health facility;
allowing the connection between any health facility with any other health facility;
training a predictive computer model based on values of the health status of the patients, their personal information like gender, family history, geographic location, age . . . etc and their outcome to discover new drugs;
training a predictive computer model based on values of the health status of the patient, their personal information like gender, family history, geographic location, age . . . etc and their outcome to discover new uses of existing drugs;
training a predictive computer model based on values of the health status of the patients, their personal information like gender, family history, geographic location, age . . . etc, their personal information and their outcome to rate the existing drugs;
training a predictive computer model based on values of the health status of the patient and their outcome to rate the health professional;
training a predictive computer model based on values of the health status of the patients, their financial status, their geographic location and their outcome to recommend a suitable health professional for the patient;
training a predictive computer model based on values of the health status of the patient and their outcome to rate the health facilities;
training a predictive computer model based on values of the health status of the patients, their financial status, their geographic location and their outcome to recommend a suitable health facility for the patient;
training a predictive computer model based on values of the health status of the patient and their outcome to make artificial intelligence based diagnosis software;
training a predictive computer model based on values of the health status of the patients, their personal information like gender, family history, geographic location, age . . . etc, their personal information and their outcome to make retrospective studies;
training a predictive computer model based on values of the unique universal predefined permanent ID of the users e.g. the DNA, to understand familial relations between users.

10. The system of claim 8, wherein the health facilities and professionals are rated based upon scientific basis.

11. The system of claim 8, wherein some of the patients' data can be accessed without authorization but under law standards in case of emergency.

12. The system of claim 8, wherein the Synchronizer module allows connection between users on different medical platforms.

13. The system of claim 8, wherein the robotic surgical module allows performing remote robotic surgeries even from other countries.

14. The system of claim 8, wherein training the artificial intelligence computer model based on values of robotic surgical surgeries to assist in and perform surgeries.

15. A computer program product comprising a non transitory computer-readable storage medium containing computer program code, the computer program code when executed by one or more processors causes the one or more processors to perform operations, the computer program code comprising instructions to:
give each health-related individual a unique universal predefined permanent ID.

16. The computer program product of claim 15, wherein giving each health-related facility a unique universal predefined permanent ID;
allowing the connection between any health-related individual user with any other;
allowing the connection any health-related individual user with any health facility;
allowing the connection between any health facility with any other health facility;
training a predictive computer model based on values of the health status of the patients, their personal information like gender, family history, geographic location, age . . . etc and their outcome to discover new drugs;
training a predictive computer model based on values of the health status of the patient, their personal information like gender, family history, geographic location, age . . . etc and their outcome to discover new uses of existing drugs;
training a predictive computer model based on values of the health status of the patients, their personal information like gender, family history, geographic location, age . . . etc, their personal information and their outcome to rate the existing drugs;
training a predictive computer model based on values of the health status of the patients, their financial status, their geographic location and their outcome to recommend a suitable health professional for the patient;
training a predictive computer model based on values of the health status of the patient and their outcome to rate the health professional;
training a predictive computer model based on values of the health status of the patient and their outcome to rate the health facilities;
training a predictive computer model based on values of the health status of the patients, their personal information like gender, family history, geographic location, age . . . etc, their personal information and their outcome to make retrospective studies;
training a predictive computer model based on values of the unique universal predefined permanent ID of the users e.g. the DNA, to understand familial relations between users.

17. The computer program product of claim 15, wherein some of the patients' data can be accessed without authorization but under law standards in case of emergency.

18. The computer program product of claim 15, wherein the Synchronizer module allows connection between users on different medical platforms.

19. The computer program product of claim 15, wherein the robotic surgical module allows performing remote robotic surgeries even from other countries.

20. The computer program product of claim 15, wherein training the artificial intelligence computer model based on values of robotic surgical surgeries to assist in and perform surgeries.
